SEQUENCE LISTING (2 sequences)

SEQ ID NO: 1 - DNA, 21 bp, artificial sequence
  sequence of first strand, may be conjugated
gtgtcgtgcctccgtgctgtg

SEQ ID NO: 2 - DNA, 21 bp, artificial sequence
  sythetic DNA, could be conjugated
cacagcacggaggcacgacac

(19) United States
(12) Patent Application Publication
(10) Pub. No.: US 2022/0047715 A1
(43) Pub. Date: Feb. 17, 2022

(54) FORCE-DEPENDENT DRUG RELEASE SYSTEM TO ENHANCE SELECTIVE KILLING AND MINIMIZE ADVERSE EFFECTS IN CANCER TREATMENT

(71) Applicant: THE JOHNS HOPKINS UNIVERSITY, Baltimore, MD (US)

(72) Inventors: Yun Chen, Pikesville, MD (US); Seungman Park, Baltimore, MD (US)

(21) Appl. No.: 17/275,341

(22) PCT Filed: Sep. 11, 2019
(86) PCT No.: PCT/US2019/050648

§ 371 Date: Mar. 11, 2021

Related U.S. Application Data
(60) Provisional application No. 62/729,768, filed on Sep. 11, 2018.

(51) Int. Cl. A61K 47/68; A61K 47/54

(52) U.S. Cl. CPC A61K 47/6849; A61K 47/6857; A61K 47/6829; A61K 47/549

(57) ABSTRACT

The disclosure provides a force-dependent drug release system. The system is configured such that the drug is only released and subsequently internalized by cancer cells, which exert at least a threshold amount of force on a DNA component of the system. The system includes a tension sensor that is used to release a chemotherapeutic agent selectively into cancer cells. The system includes a first nucleic acid single strand of DNA or DNA analog that is conjugated to a substrate, and a second nucleic acid single strand of DNA or DNA analog that is hybridized to the first single strand. The second single strand is conjugated to a cytotoxic molecule that includes a cell surface receptor ligand and a chemotherapeutic agent. The second single strand is not conjugated to the substrate. Also provided are cancer cells that display a surface receptor ligand that is bound to the cytotoxic molecule. Also provided are one or more cancer cells that have internalized a single strand conjugated to the cytotoxic molecule, but have not internalized the first strand. Also provided are methods of treating cancer by administering the system The disclosure also provides a method for treating cancer by administering to an individual in need thereof. Also provided is a method for screening or testing chemotherapeutic agents for use in the system.

### CROSS-REFERENCE TO RELATED APPLICATIONS

This application claims priority to U.S. provisional patent application No. 62/729,768, filed Sep. 11, 2018, the entire disclosure of which is incorporated herein by reference.

### BACKGROUND

Programmed cell death protein 1 (PD1) and CTLA4 have been identified to be associated with immunosuppression during tumor progression in multiple types of cancer, including melanoma, breast cancer, lung cancer and osteosarcoma. It has been demonstrated that blocking CTLA4 may enhance anti-tumor responses. It has been shown that using an antibody to block CTLA4 can restore the body's natural immunity against metastatic melanoma. Moreover, it is found that higher counts of cytotoxic T cells are in tumors after CTLA4 protein is blocked and that this results in increased killing of cancer cells and a reduction in tumor sizes. Drugs blocking CTLA4 (Ipilimumab) and PD1 (Nivolumab, Pembrolizumab, Avelumab) have been approved by FDA in recent years. The efficacy of treatment by these drugs, known as immunotherapy, has been studied in multiple clinical trials. In several clinical trials, immunotherapies involving blocking CTLA4 by Ipilimumab prolonged the survival of cancer patients compared to the control group. In one study, it was concluded that 1-year progression-free survival rate was higher with the CTLA4-blocking treatment (30.9%) than with the other chemotherapy (17.0%). Despite the high efficacy of Ipilimumab therapy, there are high rates of adverse events. Serious adverse events were experienced by 50% -55% of patients who received combined immunotherapies of Nivolumab and Ipilimumab. For patients who received Ipilimumab alone, it was concluded from 81 reports, with a total of 1265 patients from 22 clinical trials, that 72% of patients experience skin lesions (rash, pruritus, and vitiligo), colitis, hepatitis, hypophysitis, thyroiditis, and sarcoidosis, uveitis, Guillain-Barré syndrome, immune-mediated cytopenia and polymyalgia rheumatic/Horton. In one study, 85% patients receiving Ipilimumab experience adverse events. In some case reports, such adverse events result in severe complications, for example, intestinal perforation and colectomy, with fatal outcome.

It is considered that insufficient specificity is the main reason that many patients treated by the CTLA4 blocking drug Ipilimumab experience AEs. Both normal cells and cancer cells expressing CTLA4 are targeted by Ipilimumab. Normal cells expressing CTLA4 include regulatory T (T_{reg}) cells, peripheral blood mononuclear cells, B cells, CD34+ stem cells, and granulocytes. These normal cells are affected by the toxicity of the drug also. Intravenous administration of Ipilimumab further compounds the issue of adverse effects. Ipilimumab circulates the body after intravenous injection, and the normal cells expressing CTLA4 in the patient's body are affected systemically. Thus, there is an ongoing and unmet need to improve available anti-cancer approaches. The present disclosure is pertinent to this need.

### SUMMARY

The present disclosure provides a force-dependent drug release system. The system is configured such that the drug is only released and subsequently internalized, by cancer cells which exert a threshold amount of force on a DNA component of the system. Thus, the disclosure provides a tension sensor that is used to release, for example, a chemotherapeutic agent, selectively into cancer cells. 1

In one embodiment, the disclosure provides double stranded DNA, or double stranded DNA analogs, which comprise:

i) a first nucleic acid single strand of DNA or DNA analog, wherein the first single strand is conjugated to a substrate, for example, GTG TCG TGC CTC CGT GCT GTG-biotin (SEQ ID NO:1); and

ii) a second nucleic acid single strand of DNA or DNA analog, that is hybridized to the first single strand, wherein the second single strand is conjugated to a cytotoxic molecule that comprises a cell surface receptor ligand and a chemotherapeutic agent, and wherein the second single strand is not conjugated to the substrate. In embodiments, the first strand, the second strand, or both strands comprise a DNA analog, for example, toxin-CAC AGC ACG GAG GCA CGA CAC (SEQ ID NO:2).

In embodiments, the cytotoxic molecule comprises a fusion protein. In embodiments, the cell surface receptor ligand and/or the chemotherapeutic agent comprises a polypeptide. In embodiments, the cell surface receptor ligand and the chemotherapeutic agent are comprised by a contiguous polypeptide. The cell surface receptor is any cell surface receptor that can bind with specificity to a surface receptor on a cancer cell. In non-limiting examples, the cell surface receptor ligand is Cytotoxic T-Lymphocyte-Associated Antigen-4 (CTLA4) or Programmed cell death protein 1 (PD-1).

In embodiments, the chemotherapeutic agent is a toxin. In embodiments, the substrate comprises a biocompatible material.

In embodiments, the first and second strands of the dsDNA/DNA analog are separated from one another by binding of the cytotoxic molecule to a cell surface via binding of the cell surface receptor ligand to a cell surface receptor expressed by the cancer cell. Binding of the cytotoxic molecule to a cell surface via binding of the cell surface receptor ligand to the cell surface receptor on a non-cancer cell does not separate the first and second strand. In embodiments, the first and second strands can be separated from one another by application of force to the composition comprising not less than any one of 30-60 piconewton (pN).

The disclosure includes cancer cells comprising a surface receptor ligand that is bound to the cytotoxic molecule. The disclosure also includes one or more cancer cells that have internalized a single strand conjugated to the cytotoxic molecule, but has not internalized the first strand.

The disclosure also provides a method for treating cancer by administering to an individual diagnosed with or suspecting of having the cancer an effective amount of a composition that contains the first and second strands, with conjugations and a substrate.

In another aspect, the disclosure provides a method for testing a chemotherapeutic agent, the method comprising providing a composition that contains the first and second strands, with conjugations and a substrate, exposing cancer cells to the composition, and measuring killing of cancer cells subsequent to exposing the cancer cells to the composition, wherein killing of the cancer cells indicates the chemotherapeutic agent is suitable for use in treating an individual with the composition. In embodiments, the cancer cells are obtained from an individual who is diagnosed with or suspected of having a cancer.

### BRIEF DESCRIPTION OF THE FIGURES

FIG. 1: Immunostaining of cytoplasmic CTLA4 in normal cells (MCF10A and EPH4-EV) and breast cancer cells (MDA-MB231 and EO771). Scale bar: 10 μm.

FIG. 2: DNA-based tension gauge tethers (TGTs) for force measurement. (a) The CD80-Fc-Protein G-conjugated TGTs labeled with Cy3 are immobilized on glass surface to interact with cells. When the tension exceeds the specific values, depending on the immobilized position of the TGT (inset), rupture occurs and a loss of signal (dark pixels) event is recorded. (b) Using micropillar-based traction force assays, breast cancer MDA-MB231 cells exhibit 2 times higher forces than the normal MCF10A cells. (c) The images in TGT fields showed although normal cells express CTLA4, high mechanical forces are not generated to cause ruptures. Scale bar: 20 μm.

FIG. 3: Micropillar-based measurement for CTLA4-CD80 tension. (a) A cancer cell is cultured on micropillars. The forces transmitted to the CTLA4-CD80 bond bend the micropillars. (b) Cancer cells can generate ˜2-fold higher stress than normal cells. Myosin II inhibitor Blebbistatin does not inhibit the force generation transmitted through the CTLA4-CD80 bond. (c) Cancer cells generate 4-fold higher stress transmitted to the CTLA4-CD80 bond compared to integrin-fibronectin bond. Scale bar: 5 μm.

FIG. 4: Design of a representative force-dependent drug release system. After surgical removal of the primary tumor, the drug repository will be placed close to the tumor. A representative form of the repository is a tubular structure known as shunt guided into a large vein through catheters. The system comprises at least three parts: The drug to bind to cancer cells, tension sensors for force-dependent drug release, and a repository for drug storage and immobilization. The tension sensors will only rupture and release the drug protein to cancer cells capable of generating forces above the rupture threshold.

FIG. 5: CTLA4 can be detected in the cartilage tissue of a pediatric osteosarcoma patient. The cartilage tissue was surgically removed from the patient. The brightfield, nuclear staining, CTLA4 staining and the merged images are shown (panels a, b, c, d) respectively. Scale bar: 10 μm.

FIG. 6: 3D printing of biomimetic bone tissues for drug tests. The 3D bioprinter (a) has a syringe-based printing head. The enlarged view of the syringe (indicated by the arrow) is shown at the top of the right panel, (b) dispensing cell-embedded bioink. Arbitrary shapes of bone tissue, in this case a grid-like structure made of printed bone cells (c), can be printed with cells encapsulated (d). Scale bar: 20 μm.

FIG. 7. Breast cancer cells internalize immunity-activating co-receptors CD80 via a force dependent process. Normal MCF10A cells express CTLA4, which binds to CD80 on the surface of T cell stimulator cells (TCS-CD80) (a), but do not internalize the CD80. The dotted line marked the perimeter of the TCS-CD80. Breast cancer MDA-MB231 cells express CTLA4, and internalize the CD80 (b). Inhibition of force generation in MDA-MB231 cells suppress the CD80 internalization (c).

FIG. 8. Figure: Breast cancer cells suppress T cell activation via a force dependent process. After co-incubation with normal MCF10A cells, T cell stimulator cells (TCS-CD80) activate 19.4% of the resting T cells, where transcription facilitated by NF-κB and NFAT is enhanced (top). TCS-80 cells co-incubated with breast cancer MDA-MB231 loses the capacity of T cell activation by around 50% (center). TCS-80 cells co-incubated with breast cancer MDA-MB231 treated with the inhibitor of force generation activate 73.6% of the resting T cells (bottom).

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various changes may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

The disclosure includes all amino acid and polynucleotide sequences described herein, their complementary sequences, and reverse complementary sequences. The disclosure includes sequences that share sequence identity with the described sequences, provided the intended function of the molecule comprising or consisting of such sequences is maintained.

Unless otherwise indicated, nucleic acids are written left to right in 5′ to 3′ orientation; amino acid sequences are written left to right in amino to carboxyl orientation.

Any size measurement described herein can be provided as, for example, an average. Non-limiting examples include average diameter, length, width, height, average particle diameter, or may be a measure of a size distribution, such as a particle diameter distribution.

In embodiments, the disclosure provides a force-dependent drug release system. In embodiments, the drug is only released and subsequently internalized, by cancer cells which exert a threshold amount of force on a component of a composition, as further described herein. Thus, in certain implementations the disclosure provides a tension sensor that is used to release, for example, a chemotherapeutic agent, selectively into cancer cells.

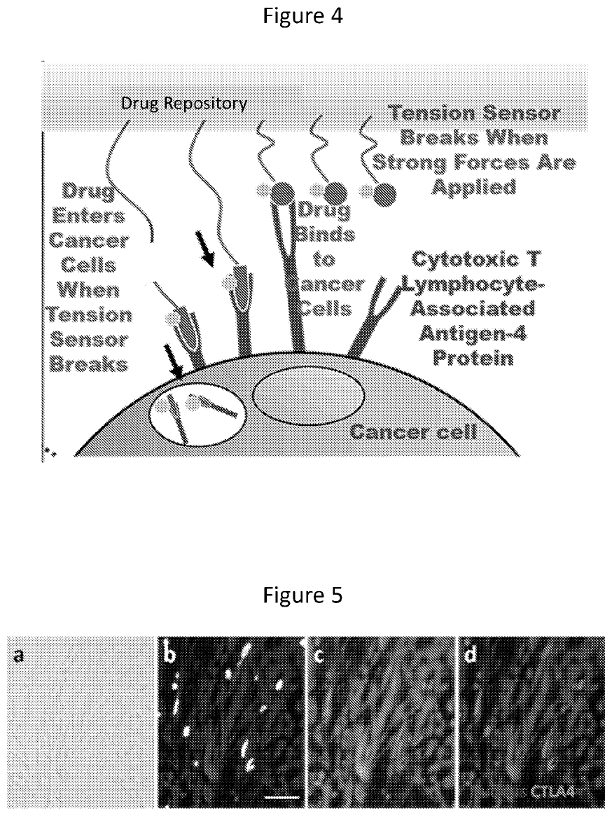

The disclosure is based in part on the discovery that high force generation on cell surface receptor/ligand complexes is a signature of many cancer cells, which is demonstrated herein using metastatic breast cancer cells. In particular, and without intending to be bound by any particular theory, it is considered this is the first demonstration that high mechanical forces generated by cancer cells are required for the ligand-mediated immunosuppression, which is demonstrated using CTLA4, as described further below. Thus, the drug release system is of this disclosure is designed to minimize the adverse effects, and improve the treatment outcomes and life quality of cancer patients, by selectively introducing a component of the composition into only cancer cells. Further, data presented herein indicate that suggest both T_{reg} cells and CTLA4-positive breast cancer cells facilitate force-dependent immunosuppression. Thus, in embodiments, the disclosure provides for specifically targeting these cell types, and restoring anti-tumor immunity, leading to better outcomes. A non-limiting example of an embodiment of this disclosure is provided in FIG. 4.

In embodiments, the disclosure provides a composition comprising:

i) a first nucleic acid single strand of DNA or DNA analog, wherein the first single strand is conjugated to a substrate, and

ii) a second nucleic acid single strand of DNA or DNA analog, that is hybridized to the first single strand, wherein the second single strand is conjugated to a cytotoxic molecule that comprises a cell surface receptor ligand and a chemotherapeutic agent, and wherein the second single strand is not conjugated to the substrate.

The terms “first” and “second” strand as used herein are arbitrary and are used for convenience to refer to a partially or fully double stranded DNA complex.

In general, the first strand and second strand will have the same or similar length of nucleotides, or modified nucleotides, such that they can hybridize to each other, such as hybridization under physiological conditions, with any desired degree of stringency. In embodiments, the first and second strand are the same length. In embodiments, the strands are from 10-100 bases, inclusive, and including all integers and ranges of integers there between. In embodiments, the strands are not more than 100 bases. In embodiments, the strands are equal to, or are not more than 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nucleotides.

In embodiments, the base content of one or both strands is modified according to a particular desired function. For example, without intending to be bound by any particular theory, it is considered that the tension exerted by cancer cells on receptor/ligand pairs as described herein can be determined for any particular type of cancer cell receptor or ligand, and this can be done in a personalized approach. For instance, a biological sample from an individual can be analyzed to assess its surface receptor, and a calculation can be made to determine a particular force that will be required to separate the second nucleic acid strand that includes the cytotoxic molecule such that the second strand and its cargo can be internalized into the cancer cells. In embodiments, a threshold force is calculated. In embodiments, the threshold force is determined to be from 30-60 piconewton (pN), inclusive and including all integers and ranges of integers there between. Such forces can be determined using approaches known in the art and adapted for use in embodiments of this disclosure, such as by using an atomic force microscope, and/or molecular tweezers, non-limiting examples of the latter are provided herein. Likewise, the base content of the first and second nucleic acid strands can be deliberately designed to account for threshold force requirements, such as by determining melting temperature, GC/AT content, and the like. Likewise, a biological sample can be tested from an individual patient to determine whether or not a cancer the individual has expresses the receptor for the particular ligand, and as such, personalized cancer treatments are included in the disclosure.

In embodiments, the first strand, or second strand, or both first and second nucleic acid single strands, may comprise or consist of a DNA analog. The DNA analog may include modified nucleotides and/or modified nucleotide linkages. In embodiments, only some nucleotides are modified, or all nucleotides are modified. Suitable modifications and methods for making DNA analogs are known in the art. Some examples include but are not limited to polynucleotides which comprise modified ribonucleotides or deoxyribonucleotide. For example, modified ribonucleotides may comprise methylations and/or substitutions of the 2′ position of the ribose moiety with an ——O—— lower alkyl group containing 1-6 saturated or unsaturated carbon atoms, or with an ——O—aryl group having 2-6 carbon atoms, wherein such alkyl or aryl group may be unsubstituted or may be substituted, e.g., with halo, hydroxy, trifluoromethyl, cyano, nitro, acyl, acyloxy, alkoxy, carboxyl, carbalkoxyl, or amino groups; or with a hydroxy, an amino or a halo group. In embodiments modified nucleotides comprise methyl-cytidine and/or pseudo-uridine. The nucleotides may be linked by phosphodiester linkages or by a synthetic linkage, i.e., a linkage other than a phosphodiester linkage. Examples of inter-nucleoside linkages in the polynucleotide agents that can be used in the disclosure include, but are not limited to, phosphodiester, alkylphosphonate, phosphorothioate, phosphorodithioate, phosphate ester, alkylphosphonothioate, phosphoramidate, carbamate, carbonate, morpholino, phosphate triester, acetamidate, carboxymethyl ester, or combinations thereof. In embodiments, the DNA analog may be a peptide nucleic acid (PNA).

The first single nucleic strand can be conjugated to a substrate using any of a wide variety of approaches, chemistries, and reagents that will be apparent to those skilled in the art, given the benefit of the present disclosure. Further, one or both strands can be functionalized with any suitable functional group, such as with an amino group. The conjugation can be at the 5′ or 3′ end, provided the strands can hybridize to one another. Conjugation can be reversible or irreversible. Moieties conjugated to the nucleic acids may be the same for each strand, or may be distinct moieties.

In embodiments, the first DNA strand or DNA analog is conjugated to a biocompatible material, which is considered to be a substrate. Such materials should be stable under physiological conditions. In embodiments, silicone is used. In embodiments, lipid-stabilized micro and nanoparticles can be used. In embodiments, a substrate used herein may comprise poly(lactide-co-galactide) (PLGA), poly(glycolide) (PGA), poly(L-lactide) (PLA), or poly(beta-amino esters). In embodiments, the compositions of this disclosure can be conjugated to a structured substrate, such as micropillars, or microneedles. Suitable micropillars and microneedles are known in the art, and can be made and adapted for use in embodiments of the present disclosure. The micropillars, microneedles, or particles may be at the micron scale (e.g., having one or more dimensions of 20-200 μm, inclusive and including all integers and ranges of integers there between). In embodiments, the substrate is selected based on having a size large enough such that it cannot be internalized by cancer cell or non-cancer cells. Thus, in certain aspects, a substrate comprising a diameter or the shortest side length of at least 100 μm is used. In embodiments, compositions of the disclosure are provided as a component of particles or hollow tubing, porous foams, or any other biocompatible materials of arbitrary shapes that can provide adequate surface area to accommodate the adequate dosage of the immobilized drug.

In non-limiting embodiments and to demonstrate a principle of the invention, a tension sensor as described herein is functionalized such that it comprises complementary nucleic acid strands functionalized with an amino group at one 5′ end; whereas the 5′ end in the other strand may be biotinylated. A streptavidin-coated substrate can be used in this configuration, but any other binding partners may be used according to the same approach.

The second nucleic acid single strand of DNA or DNA analog is conjugated to the cytotoxic molecule that comprises a cell surface receptor ligand and a chemotherapeutic agent using any of a wide variety of approaches, chemistries, and reagents that will be apparent to those skilled in the art, given the benefit of the present disclosure. There are numerous methods for conjugating chemotherapeutic agents to DNA and DNA analogs, and can be adapted for embodiments of the invention by those skilled in the art.

In certain embodiments, the cell surface receptor ligand involved in this disclosure is a ligand that binds to a cell surface protein that is expressed by cancer cells. The cell surface receptor may be expressed exclusively by cancer cells, or its expression may be upregulated in cancer cells, or it may be expressed at similar levels to non-cancer cells, which is expected to be compensated for by the tension-sensor based release approach that is described herein. In certain embodiments, the cell surface ligand is a chemokine. In embodiments, the cell surface receptor functions at least in part as an immune checkpoint protein. In embodiments, the receptor is CD80, and thus the ligand comprises Cytotoxic T-Lymphocyte-Associated Antigen-4 (CTLA4). In embodiments, the ligand binds to the cell surface receptor Programmed cell death protein 1 (PD-1), and thus may comprise an antibody or antigen-binding fragment thereof that binds with specificity to the PD-1. In embodiments, the ligand may comprise Programmed death-ligand 1 (PD-L1) or Programmed cell death 1 ligand 2 (PD-L2), and thus may comprise an antibody or antigen-binding fragment thereof that binds with specificity to the PD-L1 or PD-L1. In non-limiting embodiments, anti-PD-1 agents include Pembrolizumab and Nivolumab. An anti-PD-L1 example is Avelumab. An anti-CTLA-4 example is Ipilimumab. Compositions comprising more than one type of cell surface receptor ligand are included with this disclosure. In embodiments, the ligand may bind to any surface molecule(s) onto which cells can exert forces to accomplish endocytosis.

In embodiments, and as discussed above, the second single strand is conjugated to a cytotoxic molecule that comprises a cell surface receptor ligand and a chemotherapeutic agent. The chemotherapeutic agent is not particularly limited, so long as it capable of being internalized into the cells, or otherwise killing or restricting growth and/or proliferation of the cells. In embodiments, the chemotherapeutic agent comprises an anti-cancer small molecule. In embodiments, the chemotherapeutic agent comprises a platinum-based agent, such as carboplatin, or a cytoskeletal drug that targets, for example, tubulin, such as paclitaxel, or a DNA intercalating agent, such as doxorubicin, or a kinase inhibitor.

In embodiments, the chemotherapeutic agent comprises a polypeptide. In embodiments, the polypeptide comprises a peptide or a protein. In embodiments, the cell surface ligand and the chemotherapeutic agent are present in a contiguous polypeptide, i.e., a fusion protein, such as a protein translated from the same open reading frame. Thus, in embodiments, the surface ligand comprises a first segment, and the chemotherapeutic comprises a second segment, of a single polypeptide. Either segment can appear in any region of the polypeptide, i.e., the N-terminal region, the C-terminal region, or within the polypeptide. The polypeptide can be configured such that it can be enzymatically processed once internalized by the cells. For example, the chemotherapeutic agent may be provided with one or more proximal protease recognition sites so that it can be cleaved out of the polypeptide once internalized, such as by an intracellular protease. Thus, the chemotherapeutic segment of the polypeptide that also comprises the surface receptor ligand can be provided as a type of pro-drug that is activated, or become more effective, once it has been cleaved. In embodiments, the polypeptide comprises an immunoglobulin, such as a monoclonal antibody, or antigen-binding fragment thereof. In embodiments, the chemotherapeutic agent comprises a toxin. Non-limiting examples of suitable toxins will be apparent to those skilled in the art, and include, for example, Pseudomonas aeruginosa Exotoxin (PE), such as PE A chain, diphtheria A chain, nonbinding active fragments of diphtheria toxin, ricin A chain, abrin A chain, modeccin A chain, alpha sarcin, Aleurites fordii proteins, dianthin proteins, and Phytolaca americana proteins (PAPI, PAPII, and PAP-S), and other toxins.

Compositions of this disclosure may be provided as pharmaceutical formulations. The form of pharmaceutical preparation is not particularly limited, but generally comprises a composition as described herein, and at least one inactive ingredient. In certain embodiments suitable pharmaceutical compositions can be prepared by mixing any one type of an agent described herein, or combination of distinct agents, with a pharmaceutically-acceptable carrier, diluent or excipient, and suitable such components are well known in the art. Some examples of such carriers, diluents and excipients can be found in: Remington: The Science and Practice of Pharmacy (2005) 21st Edition, Philadelphia, Pa. Lippincott Williams & Wilkins, the disclosure of which is incorporated herein by reference.

In embodiments, compositions of this disclosure are administered to an individual in need thereof. In embodiments, the individual has been diagnosed with, is suspected of having, or is at risk of developing, any type of cancer. In embodiments, the disclosure is used for prophylaxis/and or therapy of any cancer. In embodiments, the individual is in need of treatment and/or prophylaxis of any cancer that is: breast cancer, prostate cancer, colon cancer, brain cancer, lung cancer, pancreatic cancer, skin cancer including but not limited to melanoma, stomach cancer, head and neck cancer, mouth cancer, esophageal cancer, bone cancer, ovarian cancer, colon cancer, uterine cancer, endometrial cancer, testicular cancer, bile duct cancer, bladder cancer, laryngeal cancer, thyroid cancer, retinoblastoma, any sarcoma and any carcinoma. In embodiments, the individual has blood cancer, including but not limited to any leukemia, lymphoma, or myeloma.

Compositions of this disclosure can be administered using any suitable method, device, and route. In embodiments, the compositions are administered to an individual in need thereof using parenteral, subcutaneous, intraperitoneal, intrapulmonary, intracranial, and intranasal routes. Parenteral infusions include intramuscular, intravenous, and intraarterial administrations. The compositions may be introduced directly into a tumor. In embodiments, compositions of this disclosure are provided as implantable compositions, which may be implanted directly into a tumor, or a cleared space after surgical removal of the tumor, or site integrated into vasculature close to the site where the tumor was before the surgery. For example, a repository system as described herein can be directly implanted during the operation of surgical removal of a tumor. Alternatively, the surface of a radiation catheter or medical device can be engineered to serve as a drug storage base. Thus, embodiments of this disclosure comprise an implantable drug repository, or a medical device coated with a composition described herein.

In embodiments, an effective amount of a composition described herein is administered to an individual in need thereof. In embodiments, an effective amount comprises an amount of the composition that results in any of: lethality of cancer cells, inhibition of the growth of cancer cells, inhibition in growth of a tumor, such as tumor volume, an inhibition of growth of a primary tumor, an inhibition of growth and/or formation of metastatic foci, an inhibition of metastasis, an inhibition of angiogenesis in a tumor, a stimulation of anti-cancer immune response, or an extension of life of the individual.

In embodiments, compositions of this disclosure are used concurrently or sequentially with conventional chemotherapy, or radiotherapy, or ay immunotherapy, or before or after a surgical intervention, such as a tumor resection. In embodiments, the compositions are provided only once, or weekly, monthly, every 3 months, every 6 months, yearly, or in a pre-determined interval of years.

In embodiments, a method of this disclosure comprises exposing cancer to a composition of this disclosure in vitro.

Any aspect of this disclosure can comprise comparing the effects of any composition or component thereof to a suitable reference. The reference can comprise any suitable control, value or measurement, such as a standardized curve, the area under a curve, or a comparison to the effects of a composition to normal (non-cancer cells), or cells of the same cancer, but at a different stage.

In view of the foregoing, the following additional description of the invention is encompassed by this disclosure.

Because the molecular mechanism by which CTLA4 mediates immunosuppression is yet to be elucidated, it has been challenging to address the issues of adverse effects and increase the efficacy in treating cancer. Based on data presented herein, it is considered that CTLA4 regulates immune responses through a force-dependent, two-step process. Thus, the disclosure includes strategies to boost treatment efficiency and mitigate the possible adverse effects caused by CTLA4 blockade. As such, targeting CTLA4 as described further below provides a non-limiting demonstration or one embodiment of the disclosure, and it is expected that the same approach can be extended to other cancer cell markers, as otherwise described herein.

With respect to CTLA4, it is abundantly expressed on regulatory T (T_{reg}) cells. Recently, CTLA4 overexpression has also been observed in various cancer cells of breast carcinoma, melanoma, neuroblastoma, rhabdomyosarcoma and osteosarcoma, and neoplastic lymphoid and myeloid cells. In addition, CTLA4 is also detected in normal cells other than T cells, such as peripheral blood mononuclear cells (PBMCs), B cells, CD34+ stem cells, and granulocytes. CTLA4 binds to the costimulatory receptor CD80 on antigen presenting cells (APCs), competing with CD80's other binding partners, CD28, which is expressed on conventional T (T_{conv}) cells. Binding between CD80 and CD28, in addition to TCR binding to its specific antigen, triggers signaling leading to T cell activation and the subsequent immune response against cancer cells. CTLA4, with higher affinity to CD80, can effectively disrupt T cell activation by blocking the CD80-CD28 bond formation. Moreover, it was reported in an overexpression system that CD80 could be internalized by the CTLA4-expressing cells through trans-endocytosis. As a result, CD80 can be depleted and T_{conv} cells will no longer be effectively activated by APCs. However, it is not clear whether the trans-endocytosis is a common phenomenon upon CTLA4-CD80 binding, or it is a unique mechanism exploited by cancer cells only to achieve immunosuppression.

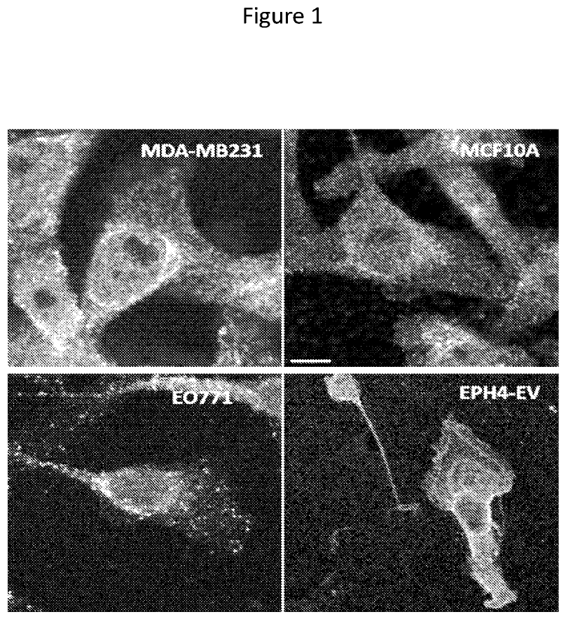

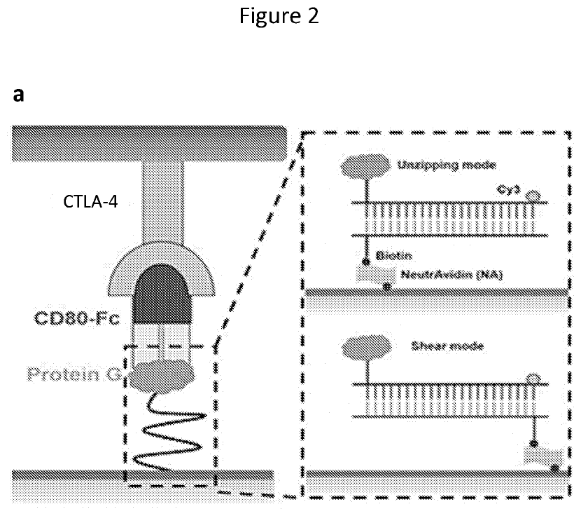

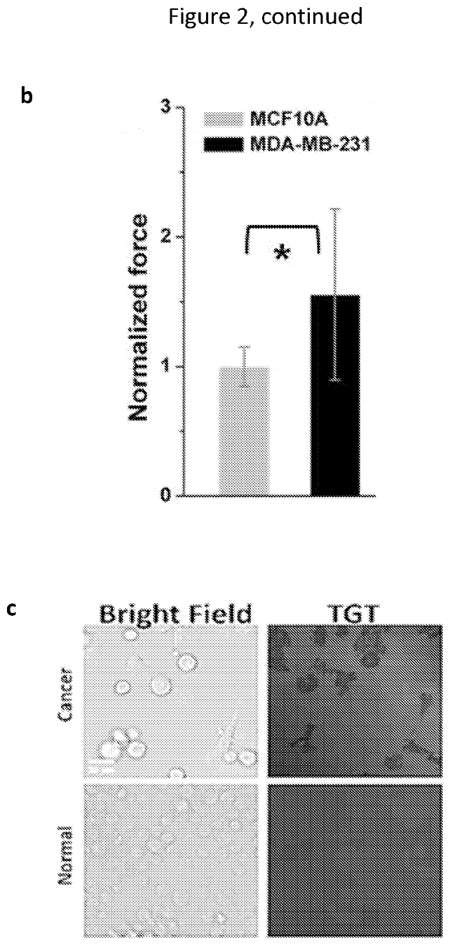

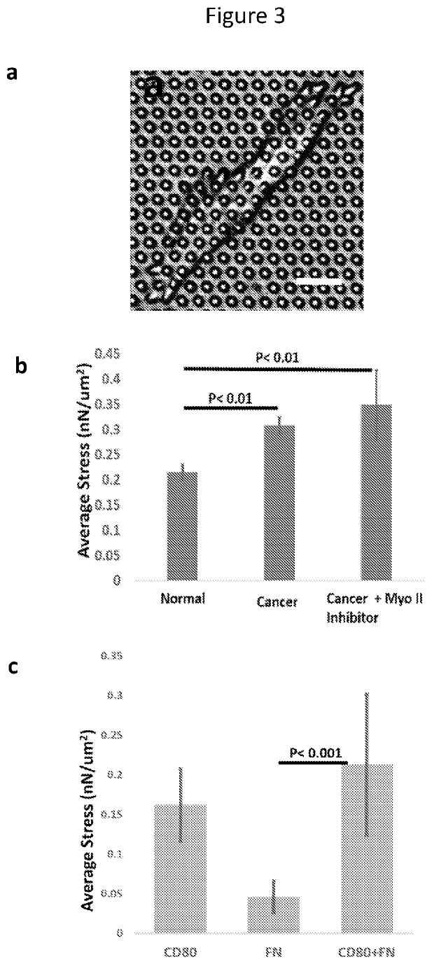

To determine which of the two possibilities described above is likely to be true, we measured the forces CTLA4 exerts on the CD80 upon binding in both normal cells and breast cancer cells. Since trans-endocytosis requires high forces to physically remove CD80 from the cell membrane of the APC, followed by internalization, we tested whether cancer cells yield higher forces if trans-endocytosis is the unique mechanism used by cancer cells to achieve immunosuppression. We tested the human breast cancer cells MDA-MB-231 and their normal counterpart MCF10A cells, as well as the mouse breast cancer cells EO771 and their normal counterpart EPH4-EV, all of which express CTLA4 (FIG. 1). The force measurement was performed by two different methods, tension gauge tether (TGT, FIG. 2a) and compliable micropillars (FIG. 3, panel a). Results from the two methods agreed with each other, where cancer cells exhibit high pulling forces via the CTLA4-CD80 bond (FIG. 2 panel b and FIG. 3 panel b) and exhibit trans-endocytosis of CD80. We also measured the forces transmitted via CTLA4 in T_{reg} cells purified from mouse spleens. The forces generated by T_{reg} cells are comparable to the ones generated by cancer cells. Moreover, when HEK293 cells, in which endogenous CTLA4 is not detected, were transfected with CTLA4 cDNA, only minimal level of forces and no trans-endocytosis of CD80 could be recorded (FIG. 2 panel c). The result indicate a specific force-generating machinery in cancer cells to facilitate trans-endocytosis, possibly leading to immunosuppression.

As a reference, we also measured the force transmitted via integrin-RGD bond, because it is extensively studied and its value has been verified by multiple techniques. Surprisingly, the force transmitted by CTLA4 is 5-fold higher than the force transmitted by integrin (FIG. 3c).

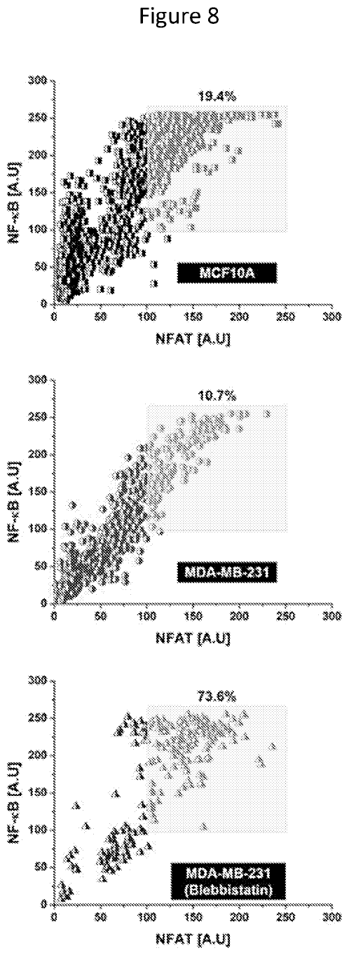

To investigate whether immunosuppression is correlated with higher forces transmitted via CTLA4-CD80 bond, T cell stimulator cells (TCS-CD80) are co-incubated with cancer cells or normal cells, and then transferred to a new plate to be co-incubated with Jurkat T cells expressing NF-κB and BFAT reporter genes, both of which are activated during immune response. After co-incubation with normal MCF10A cells, T cell stimulator cells (TCS-CD80) activate 19.4% of the resting T cells, where transcription facilitated by NF-κB and NFAT is enhanced. TCS-80 cells co-incubated with breast cancer MDA-MB231 loses the capacity of T cell activation by around 50%. TCS-80 cells co-incubated with breast cancer MDA-MB231 treated with the inhibitor of force generation activate 73.6% of the resting T cells (FIG. 8).

Without intending to be constrained by any particular theory, it is considered that two conclusions can be drawn from results presented herein. First, cancer cells can generate higher forces compared to normal cells transmitted through CTLA4-CD80 bond. Second, myosin II does not contribute to the forces exerted onto CTLA4-CD80 bond. These data support the present approach, which is based at least in part on the discovery that, despite that CTLA4 is expressed in both normal and cancer cells, only cancer cells generate sufficiently high forces, by a molecule yet to be identified, to facilitate trans-endocytosis, leading to CD80 depletion on APC surface and subsequent immunosuppression. Accordingly, the present disclosure provides in certain embodiments a novel treatment strategy that exploits the differences in the CTLA4-CD80 forces between cancer cells and normal cells, and it is expected that this discovery can be extended to other cell surface receptors, as described further above. Thus, in a non-limiting embodiment, the disclosure provides an implantable drug repository containing the fusion protein of CD80 and truncated Pseudomonas aeruginosa Exotoxin (PE). The fusion protein can be conjugated to the DNA-based tension sensor in the repository. Both normal and cancer cells expressing CTLA4 may bind to the CD80-PE fusion protein. But only when the tension exceeds the threshold, as a result of high CTLA-CD80 forces generated by cancer cells, the CD80-PE will be released from the repository and internalized by cancer cells, causing cell death. Because, in this example, the drug repository is designed with double selectivity, cell death of CTLA4-expressing normal cells with lower force generation can be minimized. It is expected that this force-dependent drug release approach can address the issues of undesired cytotoxicity, including vascular leak syndrome, asthenia, thrombocytopenia, and related conditions, which are commonly seen in immunotoxin-based therapy.

In a non-limiting embodiment, the present disclosure provides a drug release system that comprises three parts: CD80-PE to induce cytotoxicity, tension sensors for force-dependent drug release, and an implantable repository for drug storage (FIG. 4). Plasmids containing the cDNA for the fusion protein CD80-PE can be constructed by combining sequences encoded for human CD80 and the translocation and ADP-ribosylating domains of PE. The cDNA will be inserted to an appropriate plasmid for protein production by E. Coli. The tension sensor will contain double-strand PNA or DNA, other DNA analogs as described above, where the rupture forces are sufficient to separate the two strands. Suitable forces are described above, and include 54 pN, which is higher than the average forces transmitted through CTLA4-CD80 bond by normal cells, but is believed to only be achieved by cancer cells, illustrated herein using breast cancer cells. In order to assemble the repository system by conjugation, the 5′ end of one of the strands of the tension sensor will be functionalized with an amino group; whereas the 5′ in the other strand will be biotinylated. The drug repository will be fabricated using clinical grade silicone into a hollow structure, where the inner surface can be coated with CD80-PE drug through the tether of PNA-based tension sensor.

The killing efficacy of the force-dependent drug release system can be tested using, for example, a breast cancer cell line or an osteosarcoma cell line. It should be noted that high level of CTLA4 expression was detected in the cartilage tissue removed from pediatric osteosarcoma patient (FIG. 5), supporting use of the system that for treating patients.

To test the capacity of cell death induction in CTLA4-positive breast cancer cells, human breast cancer MDA-MB231 cells are incubated with the force-dependent drug release repository at appropriate bead density, a non-limiting example of which is >5×10² particles/ml, for approximately 24 hours. The rate of induced cell death is evaluated using the LIVE/DEAD cell viability assay kit (ThermoFisher). Three control experiments can be performed in parallel, including (i) MDA- MB231 cells incubated the repository system conjugated with CD80 only, (ii) MDA-MB231 cells knocked out with force-generating proteins incubated with the repository system, and (iii) normal MCF10A cells incubated with the repository system. It is expected that a significantly higher cell death rate will be observed in MDA-MB231 cells incubated with the force-dependent drug release repository, compared to the control groups.

Whether the force-dependent drug release system can reverse the immunosuppression mediated by CTLA4-positive breast cancer cells can be tested, such as by be co-incubating Jurkat T cells with MDA-MB231 cells (or any other suitable cells) and the force-dependent drug release repository, when cultured on surface, where anti-CD3 is immobilized, for 24 hours. Three control experiments can be performed in parallel, including (i) co-incubation of Jurkat T cells, MDA-MB231 cells and the repository system conjugated with CD80 only, (ii) co-incubation of Jurkat T cells, MDA-MB231 cells knocked out with force-generating proteins and the repository system, and (iii) co-incubation of Jurkat T cells, normal MCF10A cells and the repository system. The immune response is evaluated by quantifying the cytokines secreted by Jurkat T cells. It is expected that a significantly higher cell cytokine production will be observed in the co-incubation of Jurkat T cells, MDA-MB231 cells and the force-dependent drug release repository, compared to the control groups.

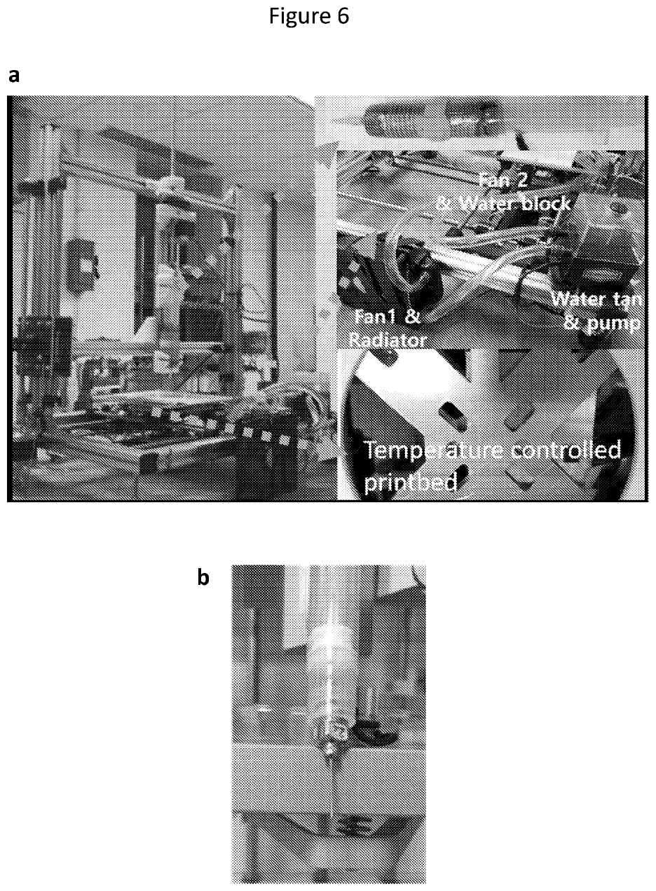

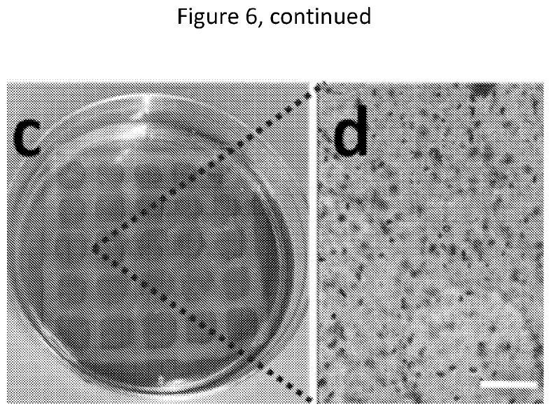

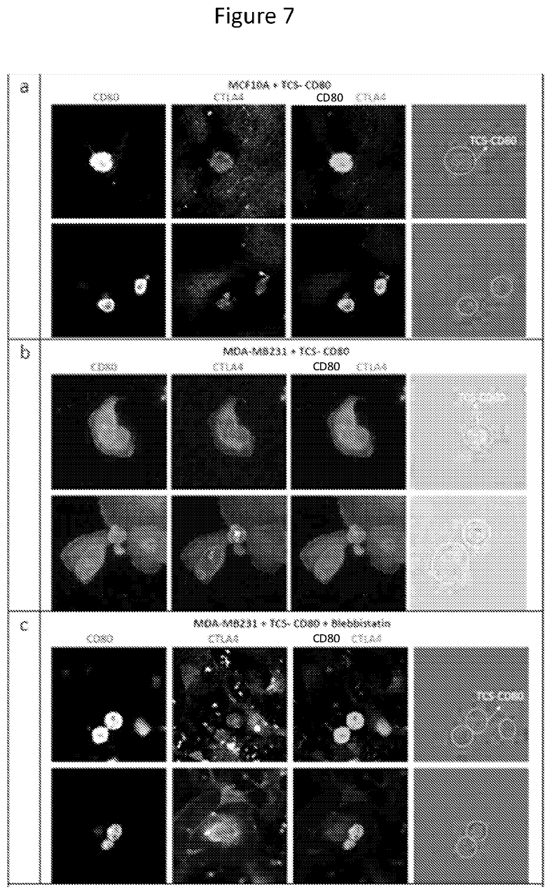

The force-dependent drug release system efficacy in killing osteosarcoma cells in a physiologically representative environment can be demonstrated. In order to recreate the physical characteristics of tumor microenvironment of osteosarcoma, the may be performed in 3D biomimetic tissue culture. Recent evidence has shown that physical factors play an important role in tumor initiation and progression as much as genetic aberrations and biochemical cues. To mimic the physical conditions in vivo, 3D culture systems are adopted to study cancer development. To obtain the results representative of physiological conditions, 3D bioprinting technology may be deployed to create 3D tissue cultures in mimicry of bones where osteosarcoma occurs, to test the efficacy of the drug release system. The stiffness, ECM organization and diffusion patterns of the bones may be recreated in the 3D printed tissue. The killing of cancer cells can be determined after incubating the drug release system with the 3D printed biomimetic bone tissue for various durations. To 3D print the biomimetic bone tissue (FIG. 6), human osteosarcoma U2OS cells are suspended in the bioink containing synthetic, osteoconductive particles (Cellink) at the density of 10⁵ cells/ml, and printed into a 3D structure. The thickness of the printed bone tissue is ˜400 μm. Given the axial resolution of the 3D bioprinter, 8 layers of 1 cm²-patches of bioink are used. To test the capacity of cell death induction in CTLA4-positive cancer cells, the 3D printed tissue will be incubated with the force-dependent drug release system for 24, 48 and 72 hours. The rate of induced cell death at each time point will be evaluated using the LIVE/DEAD cell viability assay kit (ThermoFisher). Three control experiments are performed in parallel, including (i) U2OS cells incubated the system containing immobilized CD80 only, and (ii) cancer cells with inhibited force generation by Y-27632 incubated with the system. It is expected that a significantly higher cell death rate will be observed in cancer cells incubated with the force-dependent drug release repository, compared to the control groups. The cell death can be quantitated in three experimental groups: cancer cells incubated with the drug release system, cancer cells incubated with the system containing immobilized CD80 protein only, and cancer cells knocked out with force-generating proteins incubated with the system. The second and third groups are controls. At least 10 independent repeats can be performed in each group. The results will be analyzed using Student's T test. The cell killing efficiency of the system will be deemed acceptable when the difference between the control groups and the drug-release system incubation treatment is statistically significant by at least a factor of 2, with p value less than 0.01.

There are at least two advantages using 3D printed biomimetic tissue. First, we will be able to use human cells, instead of animal cells, to test the composition. This can be extended using primary cells from patients to reflect additional clinical relevance. Second, the 3D biomimetic tissues bypass many pitfalls of 2D cell culture. For example, the lack of ECM in 2D culture diminishes crosstalk between cancer cells and the surrounding microenvironment, resulting in slower tumor progression. False positive compounds selected by drug tests in 2D culture frequently enter clinical trials, leading to high dropout rates. Thus, the disclosure includes testing the efficacy of cancer cell killing, immunity enhancement and adverse effect reduction of the force-dependent drug release system in 3D biomimetic tissue cultures using primary cells derived from patients. In particular, the efficacy of cancer cell killing, immunity enhancement and adverse effect reduction of the force-dependent drug release system can be tested using cancer cells and blood cells derived from osteosarcoma pediatric patients. Primary cancer cells removed from the patients will be used to print the 3D biomimetic tissues as described above. In order to assess the immunity enhancement effect, whole blood samples from patients will also be procured and incubated with the 3D printed tissues, along with the drug release system of this disclosure. The cancer killing efficiency will be determined by counting live and dead cells among cells expressing osteosarcoma biomarkers such as FKBP4, SRC8, PSD10, FUBP1, PARK7, NPM. The immunity enhancement efficiency will be determined by quantifying the cytokines IL-2, IL-5, IL-6, IL-7, IL-13, IFN-γ, TNF-α, MCP-1 and MIP-1β collected from the culture medium, which promote immune responses. The extent of adverse effect reduction, if any, will be determined by counting live and dead cells among cells not expressing osteosarcoma biomarkers described above.

It is expected that higher cancer cell killing rate and increased immunity will be observed in the samples incubated with the drug release system compared to the negative control, and significantly lower nonspecific toxicity on normal cells compared to the positive control. The approach of incorporating primary cancer cells and whole blood sample from patients into the testing platform is advantageous in two folds. First, this unique experimental design allows the assessment of specific killing, immune responses and adverse effects to be performed in one single integrated sample. Second, the whole blood from patients provides a more accurate physiological environment, compared to the peripheral blood mononuclear cell culture, which is the conventional subjects for immune response evaluation. The cytokine quantification obtained from the whole blood sample may give a more realistic profile of the patient's immune system.

Flow cytometry and ELISA will be performed for such evaluation. Samples from 12-20 patients may be tested. The primary cancer cells and the whole blood from each patient will be divided into three equal parts. One part will be used in the treatment groups, the other two in positive and negative controls as described above. The results will be analyzed using Student's T test. Three parameters, the specific cancer cell killing, immunity enhancement and adverse effect reduction of the drug release system, will be averaged among patients in each treatment. The drug release system will be deemed effective when the difference of each assay between the control groups and the treatment is statistically significant by at least a factor of 2, with p value less than 0.01, respectively. In addition, the variability of the three parameters will be calculated, to help understand the extent of response variation in patients.

In another embodiment, the disclosure can be used to test chemotherapeutic agents. In this approach, various chemotherapeutic agents and ligands can be tested for anti-cancer effects. Such effects include but are not limited to inhibition of cancer cell growth, and killing of cancer cells. This screening approach provides exposing cancer cells to a plurality of ligand/chemotherapeutic agents, using the system as described above. This approach can also be used to personalize a therapy, such as by using a sample obtained from an individual to determine whether any particular combination of ligand and chemotherapeutic agent has improved function, relative to another.

While the invention has been described through specific embodiments described above, some of which are prophetic, routine modifications will be apparent to those skilled in the art and such modifications are intended to be within the scope of the present invention.

1. A composition comprising:
i) a first single strand of DNA or DNA analog, wherein the first single strand is conjugated to a substrate, and
ii) a second single strand of DNA or DNA analog that is hybridized to the first single strand, wherein the second single strand is conjugated to a cytotoxic molecule, the cytotoxic molecule comprising a cell surface receptor ligand and a chemotherapeutic agent, and wherein the second single strand is not conjugated to the substrate.

2. The composition of claim 1, wherein the cytotoxic molecule comprises a fusion protein.

3. The composition of claim 1, wherein the cell surface receptor ligand and/or the chemotherapeutic agent is a polypeptide.

4. The composition of claim 3, wherein the surface receptor ligand and the chemotherapeutic agent are comprised by a contiguous polypeptide.

5. The composition of claim 4, wherein the cell surface receptor can bind with specificity to a surface receptor on a cancer cell.

6. The composition of claim 5, wherein the cell surface receptor ligand is Cytotoxic T-Lymphocyte-Associated Antigen-4 (CTLA4), Programmed cell death protein 1 (PD-1), or integrin.

7. The composition of claim 6, wherein the chemotherapeutic agent is a toxin.

8. The composition of claim 1, wherein the substrate comprises a biocompatible material.

9. The composition of claim 1, wherein the surface of the first strand, the second strand, or both strands comprise a DNA analog.

10. The composition of claim 1, wherein the first and second strands are separated from one another by binding of the cytotoxic molecule to a cell surface via binding of the cell surface receptor ligand to a cell surface receptor expressed by a cancer cell, and wherein binding of the cytotoxic molecule to a cell surface via binding of the cell surface receptor ligand to the cell surface receptor on a non-cancer cell does not separate the first and second strand.

11. The composition claim 10, wherein the first and second strands can be separated from one another by application of force to the composition comprising not less than any one of 30-60 piconewton (pN).

12. A cancer cell comprising a surface receptor ligand, wherein the cell surface ligand is bound to the cytotoxic molecule of claim 4.

13. A cancer cell that has internalized a single strand conjugated to the cytotoxic molecule of claim 1, but has not internalized the first strand.

14. A method for treating cancer comprising administering to an individual diagnosed with or suspecting of having the cancer an effective amount of a composition of claim 1.

15. A method for treating cancer comprising treating cancer comprising administering to an individual diagnosed with or suspecting of having the cancer an effective amount of a composition of claim 1.

16. The method of claim 15, wherein the the surface receptor ligand and the chemotherapeutic agent are comprised by a contiguous polypeptide.

17. The method of claim 16, wherein the cell surface receptor can bind with specificity to a surface receptor on a cancer cell.

18. A method for testing a chemotherapeutic agent, the method comprising: providing a composition of claim 1, exposing cancer cells to the composition, and measuring killing of cancer cells subsequent to exposing the cancer cells to the composition, wherein killing of the cancer cells indicates the chemotherapeutic agent is suitable for use in treating an individual with said composition.

19. The method of claim 18, wherein the cancer cells are obtained from an individual who is diagnosed with or suspected of having a cancer.

20. The method of claim 19, further comprising administering an effective amount of the composition to the individual.
